(19) United States
(12) Patent Application Publication
(10) Pub. No.: US 2024/0009142 A1
(43) Pub. Date: Jan. 11, 2024

(54) Benzaldehyde Extracted From Pit or Seeds of Rosales as a Foodstuff Preservative, Animal Feed Additive, as a Treatment for Preventing Infectious Diseases and as a Drug and Supportive Care for Treating the COVID-19 Virus

(71) Applicant: 2B-SHVAT LTD., Kfar Saba (IL)

(72) Inventors: Shmuel ROBINOV, Petach Tikvah (IL); Alex Rovinov, Petach Tikvah (IL); Avichai Simner, Ein Vered (IL); Brunia Eliezer, Hadera (IL)

(21) Appl. No.: 18/372,097

(22) Filed: Sep. 24, 2023

Related U.S. Application Data
(63) Continuation-in-part of application No. PCT/IB21/52493, filed on Mar. 25, 2021.

(51) Int. Cl. A61K 31/11; A61K 36/73; A23K 20/111; A61P 31/14; A01N 35/04; A01P 1/00; A23B 4/18

(52) U.S. Cl. CPC A61K 31/11; A61K 36/73; A23K 20/111; A61P 31/14; A01N 35/04; A01P 1/00; A23B 4/18

(57) ABSTRACT

Benzaldehyde (C₆H₅CHO), for example in a form extracted from a pit or seed of a plant of the Rosales order, in a solution of water wherein the benzaldehyde comprises at least 0.1%, and in some embodiments 3%-7% by volume of the solution, and in yet other embodiments, about 5% is used as a food preservative, an animal feed additive, a treatment and/or preventative treatment for covid-19 coronavirus infections.

### TECHNICAL FIELD

The invention relates to foodstuffs preservatives, an animal feed supplement as well as treatments for the COVID-19 virus and infectious diseases.

### BACKGROUND OF THE INVENTION

Rosales is an order of flowering plants. It contains about 7,700 species, distributed into about 260 genera. Rosales comprise nine families, the type family being the rose family, Rosaceae.

Well-known members of Rosales include: roses, strawberries, blackberries and raspberries, apples and pears, plums, peaches and apricots, almonds, rowan and hawthorn, jujube, elms, banyans, figs, mulberries, breadfruit, nettles, hops, and cannabis.

One of the substances that can be found in the pit or seed of Rosales is Benzaldehyde (C₆H₅CHO). Benzaldehyde (C₆H₅CHO) is an aromatic aldehyde bearing a single formyl group with an almond odor. It appears as a clear colorless to yellow liquid with a bitter almond odor.

### SUMMARY OF THE INVENTION

The invention provides novel uses of benzaldehyde that may have been extracted from pit or seed extracts of Rosales or made synthetically as a substitute for foodstuffs preservatives, as well as, a substitute for contemporary antibiotics used for preventing infectious diseases. Additionally, using the pit or seed extracts of Rosales as a drug and supportive care for treating the COVID-19 virus.

One aspect is a method of strengthening livestock so as to prevent the livestock from becoming diseased and/or to fight off disease of the livestock, comprising feeding the livestock with animal feed or drinking water, the animal feed or drinking water being supplemented with an additive, the additive comprising a solution of benzaldehyde in water, wherein the benzaldehyde is at least 0.1% by volume of the solution, wherein the additive is added as a liquid or as a spray. In some embodiments, the benzaldehyde is between 3% and 7% by volume of the solution.

In some embodiments, the animal feed does not contain antibiotics.

In some embodiments, the benzaldehyde is an extract of a pit or seed of a plant of the taxonomic order Rosales.

In some embodiments, the benzaldehyde comprises about 5% by volume of the solution.

Another aspect is the usage of a composition having an additive that includes a benzaldehyde as a solution of benzaldehyde in water wherein the benzaldehyde is at least 0.1% by volume of the solution, for a variety of purposes. In some embodiments, the benzaldehyde is between 3% and 7% by volume of the solution. In a limited study, the inventor has found that the composition, when supplemented to livestock feed or drinking water, and then provided to livestock as food or drinking water, has shown potential to provide growth catalyst properties (for example to increase animal growth), animal pain relief properties, and animal anti-mutational properties.

Another aspect is a composition comprising livestock feed supplemented with an additive, the additive comprising a solution of benzaldehyde in water, wherein the benzaldehyde is at least 0.1% by volume of the solution, wherein the additive is in the form of a liquid or a spray. In certain embodiments, the benzaldehyde between 3% and 7% by volume of the solution.

In some embodiments, the benzaldehyde comprises about 5% by volume of the solution.

In some embodiments, the livestock feed does not include antibiotics.

In another aspect, a method of treating a person infected with a Covid-19 coronavirus, comprises administering to the person, or the person administering to themselves, a solution of benzaldehyde in water, wherein the benzaldehyde is between 3% and 7% by volume, wherein an absolute quantity of the solution is proportionate to a body weight of the person, wherein the ingesting is implemented by drinking or intravenous injection.

In some embodiments, the absolute quantity is determined such that between 1.5 to 2.5 cc of the solution is ingested by the person for each 10 kilos of a body weight of the person.

In some embodiments, the benzaldehyde is about 5% by volume of the solution.

In some embodiments, the ingesting occurs at least twice a day.

Another aspect is a method of treating a person infected with a Covid-19 coronavirus and/or preventing the person from becoming infected with the Covid-19 coronavirus, comprising administering to the person daily a therapeutically effective amount of a medicament containing a solution of benzaldehyde in water, wherein the benzaldehyde is between 3% and 7% by volume in the solution.

In some embodiments, the administering is implemented by emitting, by a vapor producing device, a mist generated from the solution of benzaldehyde in water, and wherein the emitting is provided sufficiently close to a face of the person such that the mist is configured to be inhaled by the person, the emitting occurring for at least five minutes.

In some embodiments, the benzaldehyde is about 5% by volume in the solution.

Another aspect is a kit for treating a person infected with, and/or preventing the person from becoming infected with, a Covid-19 coronavirus, comprising a vaporizer configured to hold at least 5 milliliters of liquid and configured to vaporize a liquid contents held in the vaporizer and to emit the vaporized liquid in a prescribed direction; and at least one of (i) a solution of benzaldehyde in water, wherein the benzaldehyde is between 3% and 7% by volume in the solution and (ii) benzaldehyde separate and water separate and configured to be mixed in solution so as to arrive at the solution of benzaldehyde in water in which the benzaldehyde is between 3% and 7% by volume in the solution.

Another aspect is a food preservative, comprising a solution of between 0.25% and 1.25% by volume benzaldehyde in a solution of water, for example in a spray form.

In some embodiments, the benzaldehyde comprises between 0.5% and 1% by volume of the solution.

Another aspect is a preservative packet (also referred to as a “capsule” or a “pouch”) containing a preservative material that contains benzaldehyde. The packet includes a membrane (also referred to as an “outer layer” or “casing”) formed from a permeable material, such as nylon, that allows the diffusion of benzaldehyde molecules through it. The preservative material contains a mixture of benzaldehyde and one or more base materials and comprises between 0.1% and 40% benzaldehyde by weight. In certain embodiments, the mixture is produced by mixing one or more base materials with un-diluted liquid benzaldehyde, such that the preservative material contains between 0.1% and 40% benzaldehyde by weight. In other embodiments, the mixture is produced by mixing one or more base materials with solution of benzaldehyde in other material which can be water, oil, propylene glycol, alcohol, and the like, wherein the benzaldehyde is 80% by volume. In yet other embodiments, the mixture is produced by mixing one or more base materials with a diluted solution of benzaldehyde in water, wherein the benzaldehyde is between 3% and 7% by volume of the solution, such that the benzaldehyde-based preservative material contains between 0.1% and 40% benzaldehyde by weight.

Another aspect is a food package containing a preservative, comprising a container configured to hold food, the container also containing a solution of benzaldehyde in water wherein the benzaldehyde comprises between 3% and 7% by volume of the solution.

In some embodiments, the benzaldehyde comprises about 5% by volume of the solution.

In some embodiments, the solution is segregated behind a barrier of the container until such time as the barrier is broken.

These and other features, aspects and advantages of the invention will become better understood with reference to the following drawings, descriptions and claims.

### BRIEF DESCRIPTION OF THE DRAWINGS

Various embodiments are herein described, by way of example only, with reference to the accompanying drawings, wherein:

FIG. 1A is a chart showing certified experimental microbiology test results (after 10 minutes of treatment with the solution in an ice-water environment) of the use of a composition on fish, in accordance with one embodiment;

FIG. 1B is a chart showing certified experimental microbiology test results (after 5 days of treatment with the solution) of the use of a composition on fish, in accordance with one embodiment;

FIG. 1C is a chart showing certified experimental microbiology test results (after 12 days of treatment with the solution) of the use of a composition on fish, in accordance with one embodiment;

FIG. 2 is a comparison of a particular food with, and without, a preservative that is in accordance with one embodiment;

FIG. 3 is a flow chart showing a method in accordance with one embodiment; and

FIG. 4 is a flow chart showing a method in accordance with one embodiment

FIG. 5 shows an exemplary packet, containing a benzaldehyde-based preservative material, according to an embodiment of the present invention.

FIG. 6 shows multiple preserving packets, each being implemented according to the packet illustrated in FIG. 5, in use as a food preservative to preserve raw fish, according to an embodiment of the present invention.

### DETAILED DESCRIPTION OF THE EMBODIMENTS

The following detailed description is of the best currently contemplated modes of carrying out the invention. The description is not to be taken in a limiting sense, but is made merely for the purpose of illustrating the general principles of the invention, since the scope of the invention is best defined by the appended claims.

The invention is not limited in its application to the details of construction and the arrangement of the components set forth in the following description. The invention is capable of other embodiments, or of being practiced or carried out in various ways. Also, it is to be understood that the phraseology and terminology employed herein is for the purpose of description and should not be regarded as limiting.

The invention provides novel uses of pit or seed extracts of Rosales. The pit or seed extract is extracted from Rosales, such as, raw bitter almonds, apples, peaches etc. using, for example, a standard extraction procedure used in the industry. The pit or seed extract includes a plurality of components including, for example, Benzaldehyde (C₆H₅CHO). After trying a variety of different compounds found in the pit or seed of these fruits, Applicant has concluded that Benzaldehyde (C₆H₅CHO) is effective at counteracting spoilage of food, counteracting the build-up of bacteria and even treating Covid-19.

In one embodiment, the pit or seed extract is used, for example, as a foodstuff preservative. The pit or seed extract is added to the foodstuff to prevent its decomposition by microbial growth or by undesirable chemical changes. The pit or seed extract is added in one of its different forms (e.g., solid, liquid, or gas form) and is assimilated into the foodstuff mixture.

In another embodiment, the pit or seed extract is used, for example, as a substitute for contemporary antibiotics used for preventing infectious diseases, for example, in animals such as poultry, fish, cattle and the like. The pit or seed extract is, for example, in the form of a capsule or a tablet. The capsule or tablet is given to the animal orally as part of its food much like a food supplement.

In yet another embodiment, the pit or seed extract is used, for example as medicine and supportive care for treating the COVID-19 virus. The pit or seed extract is, for example, in the form of a capsule or a tablet given to patients orally. Alternatively, the pit or seed extract is, for example, in its liquid form. In its liquid form, the pit or seed extract is given to patients through, for example, an IV (intravenous).

The principles and operation of Benzaldehyde Extracted From Pit or Seeds of Rosales as a Foodstuff Preservative, Animal Feed Additive, as a Treatment for Preventing Infectious Diseases and as a Drug and Supportive Care for Treating the COVID-19 Virus may be better understood with reference to the drawings and the accompanying description.

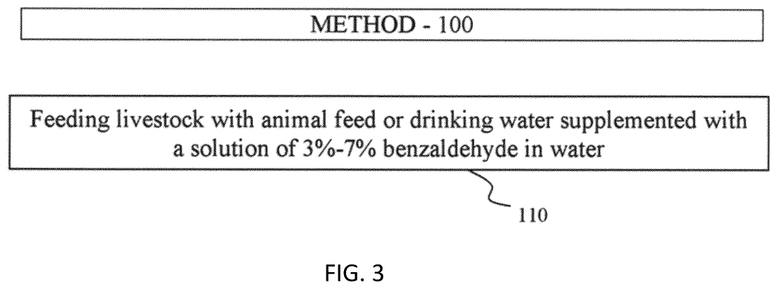

As shown in FIG. 3, one embodiment is a method 100 of strengthening livestock so as to prevent the livestock from becoming diseased and/or to fight off disease of the livestock. The method 100 comprises a step 110 of feeding the livestock with animal feed or drinking water, the animal feed or drinking water being supplemented with an additive. The additive may comprise a solution of benzaldehyde in water, wherein the benzaldehyde is at least 0.1% by volume of the solution, wherein the additive is added as a liquid or as a spray. In certain embodiments, the benzaldehyde is between about 3% and about 7% by volume of the solution. In some embodiments, the benzaldehyde is about 5% by volume of the solution. In other embodiments, the benzaldehyde is 3% or is 4% or is 5% or is 6% or is 7% by volume.

As a result of the addition of the benzaldehyde solution to the animal feed, it is no longer necessary to add antibiotics. The benzaldehyde solution is a substitute for the antibiotics since the solution is highly antibiotic and antiviral. The quantity added to the animal feed may be geared to accomplish the objectives of the substituted antibiotics. Accordingly, the animal or livestock feed to which the solution of benzaldehyde is added does not contain antibiotics in some embodiments.

The term “about X %” shall mean “X” plus or minus a tenth. For example, the phrase “about 5%” means from 4.5% to 5.5%. Similarly, the term “about X minutes” shall mean “X minutes” plus or minus a tenth. For example, the phrase “about 5 minutes” means from 4.5 minutes to 5.5 minutes.

In one implementation, the benzaldehyde used for the solution is obtained by extracting it from a pit or seed of a plant of the taxonomic order Rosales including one or more of: roses, strawberries, blackberries and raspberries, apples and pears, plums, peaches and apricots, almonds, rowan and hawthorn, jujube, elms, banyans, figs, mulberries, breadfruit, nettles, hops, and cannabis. In another implementation, the benzaldehyde used in the solution is prepared synthetically.

The additive added to the animal feed may comprise a solution of benzaldehyde in water, wherein the benzaldehyde is at least 0.1% by volume of the solution, and may be added in the form of a liquid or may be sprayed on the animal feed. In certain embodiments, the benzaldehyde between 3% and 7% by volume of the solution.

In a limited study, the inventor has found that the additive, when supplemented to livestock feed or drinking water, and then provided to livestock as food or drinking water, has shown potential to provide growth catalyst properties (for example to increase animal growth), animal pain relief properties, and animal anti-mutational properties. Thus, according to certain embodiments, there is a method of inducing growth and/or relieving pain and/or reducing likelihood of mutations in livestock. This method is similar to the method described with reference to FIG. 3, and comprises a step of feeding the livestock with animal feed or drinking water, the animal feed or drinking water being supplemented with an additive, wherein the additive comprises a solution of benzaldehyde in water, wherein the benzaldehyde is at least 0.1% by volume of the solution. In some embodiments, the benzaldehyde between 3% and 7% by volume of the solution. The additive may be added as a liquid or as a spray.

Another embodiment is a method 200 of treating a person infected with a bacterial or viral infection comprises administering to the person, or the person administering to themselves, a solution of benzaldehyde in water, wherein the benzaldehyde is between 3% and 7% by volume. The administering may be accomplished in a number of ways including having the person drink the solution or administering to the person an intravenous injection.

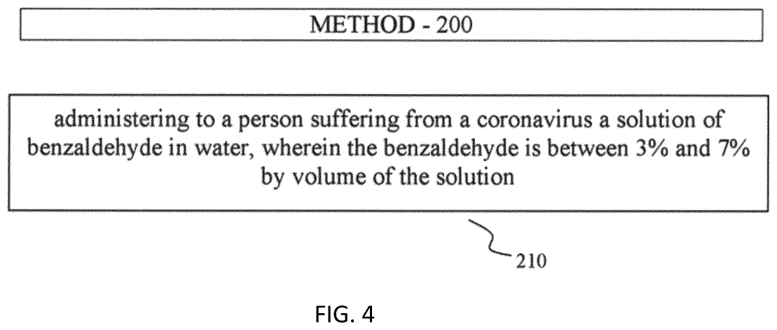

In another embodiment shown in FIG. 4, a method 200 of treating a person infected with a Covid-19 coronavirus, or a person not infected with it but who wants a protection or preventative against becoming affected, comprises a step 210 of administering to the person, or the person administering to themselves, a solution of benzaldehyde in water, wherein the benzaldehyde is between 3% and 7% by volume. The administering may be accomplished in a number of ways including having the person drink the solution or administering to the person an intravenous injection.

In some embodiments, an absolute quantity of the solution is proportionate to a body weight of the person. For example the absolute quantity may be determined such that between 1.5 to 2.5 cc of the solution is ingested by the person for each 10 kilos of a body weight of the person. For example a person who weighs 70 kilos would receive 10.5 to 17.5 cc of the solution. In some embodiments, the benzaldehyde is about 5% by volume of the solution. In other embodiments, the benzaldehyde is 3% or is 4% or is 5% or is 6% or is 7% by volume.

The administering of the benzaldehyde solution to the person may occur once in the morning and once in the evening. In other embodiments, it may occur at least twice a day or three times a day or at least three times a day at various times of day.

Another embodiment is a method 100 of treating a person infected with a Covid-19 coronavirus and/or preventing the person from becoming infected with the Covid-19 coronavirus, comprising a step 110 of administering to the person, or having the person administer to themselves, daily a therapeutically effective amount of a medicament containing a solution of benzaldehyde in water, wherein the benzaldehyde is between 3% and 7% by volume in the solution. In some embodiments, the benzaldehyde is about 5% by volume of the solution. In other embodiments, the benzaldehyde is 3% or is 4% or is 5% or is 6% or is 7% by volume.

In some embodiments, the administering is implemented by emitting, by a vapor producing device, a mist generated from the solution of benzaldehyde in water, and wherein the emitting is provided sufficiently close to a face of the person such that the mist is configured to be inhaled by the person. In one non-limiting example, the vapor producing device is a vaporizer or humidifier of the type used by parents to deliver medications or medicaments to sleeping children. In other embodiments, the vapor producing device is a device that converts a liquid into a mist or vapor and constantly emits the resulting mist or vapor that has been produced. The amount of time that the patient is situated near the vapor producing device or in the same room as the vapor producing device can vary as long as the person is configured to inhale the vapors. In some embodiments, the vapor is emitted near the person or patient or in the same room as the person or patient for at least three minutes or about 5 minutes or at least five minutes or at least 7 minutes or about 10 minutes at least ten minutes or for 10-15 minutes or at least 15 minutes or longer.

Another embodiment is a kit for treating a person infected with, and/or preventing the person from becoming infected with, a Covid-19 coronavirus. The kit may comprise a vaporizer configured to hold at least 5 milliliters of liquid and configured to vaporize a liquid contents held in the vaporizer and to emit the vaporized liquid in a prescribed direction. The kit may further comprise at least one of (i) a solution of benzaldehyde in water, wherein the benzaldehyde is between 3% and 7% by volume in the solution and (ii) benzaldehyde separate and water separate and configured to be mixed in solution so as to arrive at the solution of benzaldehyde in water in which the benzaldehyde is between 3% and 7% by volume in the solution. In some embodiments, the kit also includes instructions to a user as to how to vaporize and inhale the solution for treating and/or preventing an infectious disease, the instructions appearing on printed material or a computer disc configured to display the instructions on a display.

Another embodiment is a food preservative, comprising a solution of between 0.25% and 1.25% by volume benzaldehyde in a solution of water. In some embodiments, the benzaldehyde comprises from about 0.5% to about 1.0% of the solution, or in some other embodiments about 1.75%. In other embodiments, the benzaldehyde is 3% or is 4% or is 5% or is 6% or is 7% by volume. In some versions, the benzaldehyde solution is in the form of a spray that has been sprayed on a food item, in non-limiting examples, such as fish, meat or bread.

Another embodiment is the food preservative comprising a solution of between 0.25% and 1.25% by volume benzaldehyde in a solution of water combined with a food (such as meat, fish or bread).

Another embodiment is the food preservative comprising a solution of between 0.25% and 1.25% by volume benzaldehyde in water combined with a food container.

Another embodiments is a food package containing a preservative, comprising a container configured to hold food, the container also containing a solution of benzaldehyde in water wherein the benzaldehyde comprises between 3% and 7% by volume of the solution. In some embodiments, the benzaldehyde comprises 3% or about 3% or 4% or about 4% or 5% or about 5% or 6% or about 6% or 7% or about 7% by volume of the solution.

In some embodiments, the solution is segregated behind a barrier of the container until such time as the barrier is broken.

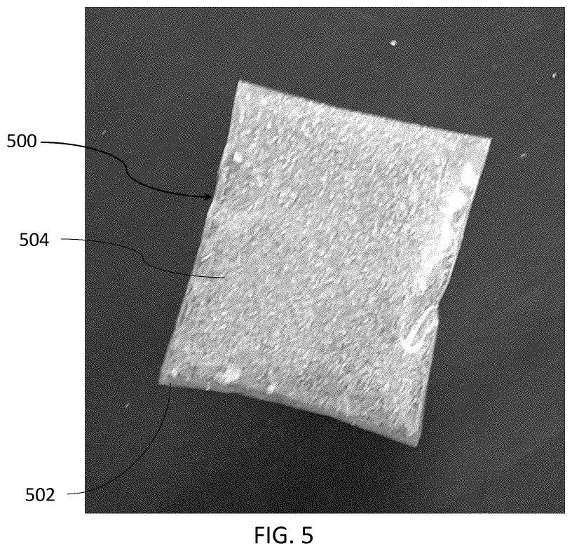

Referring now to FIG. 5, another embodiment is a perishable material preserving packet (or “capsule” or “pouch”) 500 that preserves perishable materials including foodstuffs, such as fish, eggs, cheese, vegetables, fruits, and the like, as well as perishable non-foodstuffs such as picked flowers. The packet 500 has a membrane (or “outer layer” or “casing”) 502 that contains a quantity of a benzaldehyde-based preservative material 504. The preservative material 504 is produced by mixing benzaldehyde as liquid material with one or more base material such that the preservative material 504 contains between approximately 0.1% and approximately 40% benzaldehyde by weight.

The base material is a material that absorbs liquids or gases, and can be a solid material or a semi-solid material or other type of liquid and/or gas absorbing material. A non-exhaustive list of example base materials will be provided below.

In certain embodiments the base material is sawdust.

In certain embodiments, the benzaldehyde material has a concentration of 80% active ingredient (i.e., benzaldehyde) and a remaining 20% of inactive ingredient, which can be, for example, water, oil, propylene glycol, or alcohol.

In certain embodiments, the benzaldehyde liquid material used to produce the preservative material 504 is a un-diluted benzaldehyde liquid, and the preservative material 504 is produced by combining the un-diluted liquid benzaldehyde material with the base material (or base materials), as a mixture, such that the preservative material 504 contains between approximately 0.1% and approximately 40% benzaldehyde by weight.

In other embodiments, the benzaldehyde liquid material used to produce the preservative material 504 is a diluted form of benzaldehyde liquid. For example, the benzaldehyde liquid material can be produced by combining a solution of benzaldehyde in water (referred to as “benzaldehyde solution”), and this diluted benzaldehyde liquid can be combined with the base material (or base materials) as a mixture, such that the preservative material 504 contains between approximately 0.1% and approximately 40% benzaldehyde by weight.

In other embodiments the benzaldehyde liquid material used to produce the preservative material 504 is a diluted form of benzaldehyde in other material which can be, for example, water, oil, propylene glycol, or alcohol, wherein the benzaldehyde is 80% by volume.

According to certain embodiments, the benzaldehyde is between 3% and 7% by volume of the benzaldehyde solution. In other embodiments, the benzaldehyde solution is between 0.25% and 1.25% by volume benzaldehyde in a solution of water. In yet other embodiments, the benzaldehyde comprises from about 0.5% to about 1.0% of the solution, or in some other embodiments about 1.75%.

Examples of base materials that can be combined with the benzaldehyde liquid (either diluted or un-diluted) as a mixture to produce the preservative material 504 include, but are not limited to, sand, sawdust, bran, leaves, cotton, wood chips, paper, straw, sponge, rubber, silica resin, plastic, and combinations thereof.

The prepared quantity of preservative material 504 can be placed within an opening of the membrane 502, and the membrane 502 can then be closed/sealed to form a seal around the preservative material 504 thereby maintaining the solid and/or liquid particles of the preservative material 504 therein and preventing the solid and/or liquid particles of the preservative material 504 (i.e., the mixture) from escaping and coming into direct contact with the environment.

The membrane 502 is constructed from a material that is permeable to benzaldehyde molecules, i.e., the membrane 502 allows benzaldehyde molecules to permeate therethrough, which allows benzaldehyde molecules in the preservative material 504 to escape from the packet 500 to the environment surrounding the packet 500 via the membrane 502. As a permeable structure, the membrane 502 is porous, having pore size that is sufficiently large enough to allow the diffusion of benzaldehyde molecules (emanating from the preservative material 504) through it, but small enough to prevent leakage or spilling of solid, semi-solid, or liquid particles of the mixture from the packet 500. A non-exhaustive list of materials from which the membrane 502 can be constructed from nylon, polyethylene, or any other suitable porous material. In one particularly preferred but non-limiting embodiment, the membrane 502 is constructed from nylon, which can have pore size as small as approximately 0.2 microns (μm), but can also have pore size of approximately 0.8 μm or 1.2 μm.

The packet 500 can be produced so that it is of any suitable size, and can therefore be produced to contain any suitable quantity of the preservative material 504. For example, the packet 500 can be approximately 2 centimeters wide and 2 centimeters long, with a thickness of approximately half a centimeter.

For effective use, the packet (or packets) can be preferably deployed in relative proximity to the perishable material that is to be preserved so that the benzaldehyde vapor emanating from the preservative material 504 can reach the perishable material. The distance between the packet (or packets) and the material to be preserved typically depends on various parameters, including the quantity of the preservative material, the amount of benzaldehyde in the preservative material, and the type of perishable material to be preserved. Accordingly, there may be certain situations where preservation is effective when the packet is separated from the perishable material by a distance of several meters, and other situation where preservation is effective only when the packet is in direct or indirect contact with the perishable material.

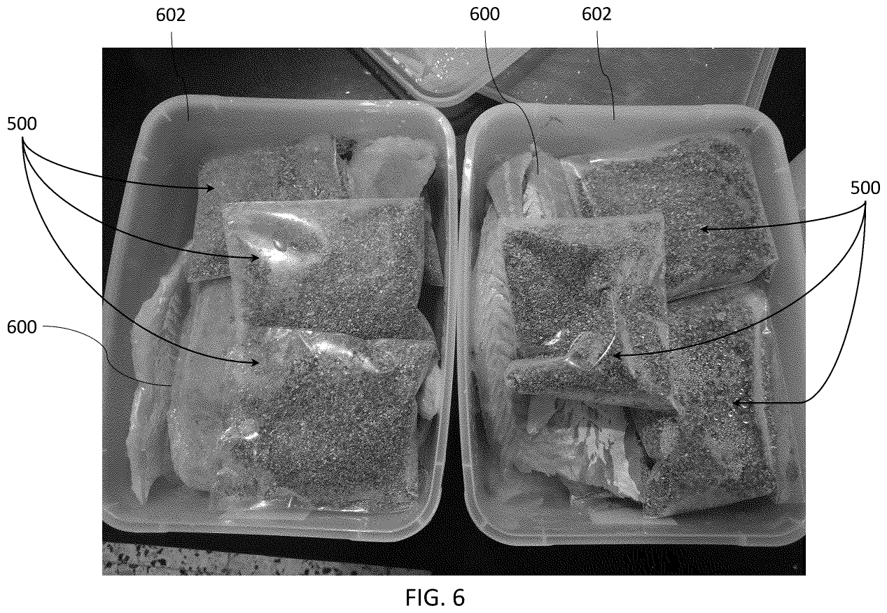

As one deployment example, the packet (or packets) can be deployed in direct contact or indirect contact with the perishable material that is to be preserved. FIG. 6 illustrates such a deployment example in which a plurality of packets 500 are in use as a food preservative to preserve pieces of raw fish 600 contained in a container 602. In the illustrated example, some of the packets are deployed in direct contact with at least some of the pieces of raw fish, and other of the packets are deployed in indirect contact with other pieces of the raw fish.

The packet according to the embodiments described herein can extend the shelf life of perishable materials by several days, and can be used to advantage in supermarkets, grocery stores, and food packing houses to extend the shelf life of foodstuffs.

### Example 1—A Laboratory Study on Fish

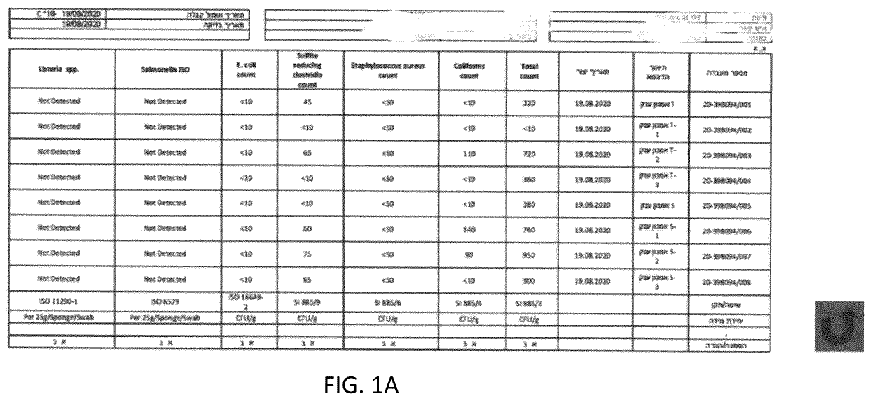

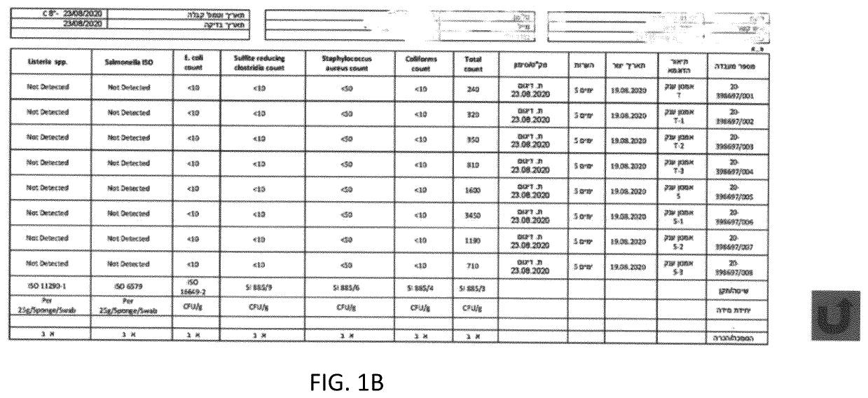

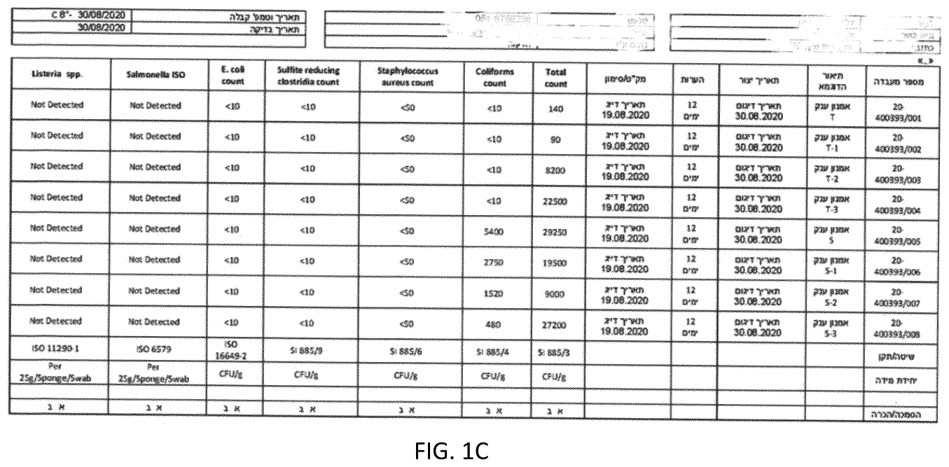

As shown in FIG. 1A, FIG. 1B, FIG. 1C, under laboratory conditions performed by the Institute for Food Microbiology in Israel during August through September 2020, a solution of formaldehyde in water (5% by volume) was poured on large fish called tilapia (the English translation of the fish that in Hebrew is called Amnoon), a freshwater fish that feeds on plant life. The fish were divided into four groups labeled “T”, “T1”, “T2” and “T3”. A corresponding amount (by weight) offish was divided into four control group and was labeled as “S”, “S1”, “S2” and “S3”. “T1”, “T2” and “T3”. Were treated with the 5% benzaldehyde solution in water. “S” and “T” were similarly treated with the benzaldehyde solution but in ice water (the benzaldehyde solution had been placed in the freezer).

The amount of specific bacteria was measured for each group of fish at three times (both with the treated group and the control group at the same time). The three times are: after 10 minutes (FIG. 1A), after 5 days of continuous treatment (FIG. 1B) and after 12 days of continuous treatment (FIG. 1C). In each case, the total bacterial count was also measured and the results appear in specific columns depending on the specific bacteria (Listeria spp., Salmonella ISO, E. coli count, Sulfite reducing clostridia count, Staphylococcus aureus count, Coliforms count, total count). The results appear in FIGS. 1A-1C.

The results show that the treated groups of fish showed marked reduction in bacteria levels. For example, following a period of 12 days from fishing to test and application of the benzaldehyde 5% preservative solution, the following results were obtained: The total count for the treated fish was 140. The total count for the control group was 29,250. For the Coliforms (measured in CFU/G) the results were <10 for the treated group and 5,400 for the control group.

The results showed a very significant reduction in bacteria for fish that were treated with the solution of benzaldehyde 5% compared to the control group.

### Example 2—A Study of Food Preservatives

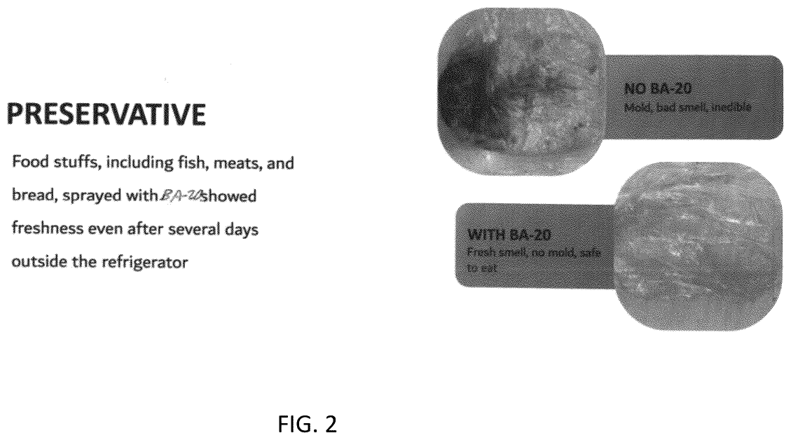

As shown in FIG. 2, under laboratory conditions, a solution of formaldehyde in water (5% by volume) was sprayed on a variety of foodstuffs including fish, meats, and bread. The foodstuffs were in ice water and were sprayed with the solution of benzaldehyde 5% in water showed freshness even after days outside the refrigerator. The same foodstuffs in ice water that were not sprayed with the formaldehyde solution were moldy, had bad smell and were inedible.

### Example 3—Observational Study

The regimen of the invention has also been observationally tested on non-hospitalized patients suffering from COVID-19 to evaluate the efficacy of a treatment regimen consisting of a solution of benzaldehyde in water with the benzaldehyde being about 5% of the solution by volume. Each patient drank the solution twice a day, for example once in the morning and once at night. The absolute amount drunk based based on body weight. For each 10 kilo of body weight the amount drunk was 2 cc. For example, a person who weighed 70 kilo drank 14 cc. The results of the observational study showed that oral administration of the liquid solution to patients suffering from COVID-19 (including fever, lung congestion, infection) stopped the progression and deterioration of moderately to severely ill patients.

In these patients, significant improvement was noted during the observation period. Specifically, within 48-72 hours, Covid 19 symptoms abated.

In a small percentage of the total number of patients, the patients were hospitalized patients but were not undergoing any regimen because they were told to wait or in a few cases were undergoing a standard regimen that they were not responding to and that continued during the administration of the benzaldehyde solution. In these patients, significant improvement was noted during the observation period. Specifically, within 48-72 hours, Covid 19 symptoms abated.

While the invention has been described with respect to a limited number of embodiments, it will be appreciated that many variations, modifications, and other applications of the invention may be made. Therefore, the claimed invention as recited in the claims that follow is not limited to the embodiments described herein.

What is claimed is:

1. A method of strengthening livestock so as to prevent the livestock from becoming diseased and/or to fight off disease of the livestock, comprising:
feeding the livestock with animal feed or drinking water, the animal feed or drinking water being supplemented with an additive, the additive comprising a solution of benzaldehyde in water, wherein the benzaldehyde constitutes at least 0.1% by volume of the solution, wherein the additive is added as a liquid or as a spray.

2. The method of claim 1, wherein the animal feed does not contain antibiotics.

3. The method of claim 1, wherein the benzaldehyde is an extract of a pit or seed of a plant of the taxonomic order Rosales.

4. The method of claim 1, wherein the benzaldehyde comprises about 5% by volume of the solution.

5. A method of treating a person infected with a Covid-19 coronavirus, comprising:
administering to the person, or the person administering to themselves, a solution of benzaldehyde in water, wherein the benzaldehyde is between 3% and 7% by volume, wherein an absolute quantity of the solution is proportionate to a body weight of the person, wherein the ingesting is implemented by drinking or intravenous injection.

6. The method of claim 5, wherein the absolute quantity is determined such that between 1.5 to 2.5 cc of the solution is ingested by the person for each 10 kilos of a body weight of the person.

7. The method of claim 5, wherein the benzaldehyde is about 5% by volume of the solution.

8. The method of claim 5, wherein the ingesting occurs at least twice a day.

9. A preservative packet comprising:
a membrane that is permeable to benzaldehyde molecules; and
a quantity of preservative material contained within the membrane, the preservative material formed as a mixture of one or more base materials with benzaldehyde, such that the preservative material contains between 0.1% and 40% benzaldehyde by weight.
